(19) United States
(12) Patent Application Publication
(10) Pub. No.: US 2024/0050026 A1
(43) Pub. Date: Feb. 15, 2024

(54) MULTI-FUNCTION DEVICE AND A MULTI-FUNCTION SYSTEM FOR ERGONOMICALLY AND REMOTELY MONITORING A MEDICAL OR A COSMETIC SKIN CONDITION

(71) Applicants: DERMATOO, Chaudfontaine (BE); IOT-D, Liege (BE)

(72) Inventors: Vincent Joassin, Comblain-au-pont (BE); Jeremie Bours, Comblain-au-pont (BE); Romain Bajoit, Annut (BE); Julien Delarbre, Embourg (BE); Maxime Noirhomme, Limerle (BE); Sebastien Blondiau, Liege (BE); Robin Murgira, La Roche-en-Ardenne (BE); Francois Delarbre, Wasseiges (BE); Francois Royen, Donceel (BE); Maxime Aerts, Retinne (BE)

(21) Appl. No.: 18/278,891

(22) PCT Filed: Jan. 24, 2022
(86) PCT No.: PCT/EP2022/051519

§ 371 Date: Aug. 25, 2023

(30) Foreign Application Priority Data
Feb. 25, 2021 (BE) BE2021/5133

(51) Int. Cl. A61B 5/00; A61B 5/01

(52) U.S. Cl. CPC A61B 5/441; A61B 5/0077; A61B 5/01; A61B 5/7425; A61B 5/0022; A61B 2560/0223

(57) ABSTRACT

A multi-function device allows for the ergonomic and remote monitoring of a skin conditions. The multi-function device captures three-dimensional image information, fluorescence information, temperature information and voice information. In a preferred embodiment, the multi-function device calibrates varying light conditions, distances between the camera and the region of interest of the skin, and angles through a calibration tube mounted on the camera of an multi-function device.

### BACKGROUND

This invention relates to a multi-function method, a multi-function system and a multi-function device for remotely monitoring a medical or a cosmetic skin condition. Monitoring of a medical or of a cosmetic skin condition remotely for multiple parameters requires several devices carrying imaging systems, temperature sensors and data transmission systems. Handling multiple devices is cumbersome. They imply multiple costs, multiple charging systems and multiple data transmission systems. The captured sensor and image data has to be transmitted and reconciled. Moreover, images of a medical or a cosmetic skin condition may vary significantly. Major variation factors include brightness, distance and image angle. Therefore, the present invention aims at providing medical and cosmetic practitioners with an integrated solution combining multiple sensors and imaging systems as well as data transmission systems. In a preferred embodiment, multi-function device aims at providing calibrated image reconstructions of medical or cosmetic skin conditions to allow for remote monitoring in an ergonomic manner and surveillance in spite of these disturbing variations.

### PRIOR ART

EP3559741 to Esthetic Visual Analytics discloses a camera comprising two imaging systems each comprising a different optical path corresponding to a different viewing angle of an object. The camera further comprises two masks comprising multiple apertures, a detector and a processor configured to produce a multi-layer three dimensional reconstruction of the object. However, neither a temperature measurement nor a calibration is described.

WO2020148726 to Moleculight describes a portable, modular handheld imaging system. The modular system comprises a first housing portion and a second housing portion. The first housing portion includes at least one excitation light source. A first filter is configured to detect and permit a passage of selected optical signals responsive to illumination with the excitation light to a first image sensor. A second filter is configured to detect and permit a passage of selected optical signals responsive to illumination of the target surface with white light to a second image sensor. The second housing portion is configured to releasably receive the first housing portion. The second housing portion includes a display and a processor configured to receive the detected fluorescent and white light optical signals and to output a representation of the target surface to the display based on the detected optical signals. Images acquired and created in the process of forming a three-dimensional fluorescent image of a target. Exemplary excitation light sources include violet/blue LEDs having a wavelength of between about 400 nm-about 450 nm, and in one example, having a wavelength of about 405 nm are disclosed. Fluorescent strips are placed into the field of view during wound imaging for calibration. Temperature are suggested for monitoring the temperature of the LEDs. However, this document does not disclose a temperature sensor for measuring the temperature of a region of interest of the skin.

EP3747022 to Moleculight discloses a computer-implemented method for wound analysis. At least one area of interest in an image is determined based on a chroma mask. The chroma mask is based on a histogram of pixel values. One or more wound characteristics are determined. The wound characteristics comprise the wound boundary, a wound size, a wound depth, a bacterial presence, a bacterial load, a wound temperature, a connective tissue presence, a blood presence, a bone presence, a change in tissue or cellular wound components, a vascularization, or a necrosis. Ultraviolet, visible, near-infrared and infrared light sources are also suggested. The device has a digital image acquisition device, such as digital camera, video recorder, camcorder, cellular telephone with built-in digital camera, ‘Smart’ phone with a digital camera, personal digital assistant (PDA), laptop/PC with a digital camera, or a webcam. However, this document does not disclose a unique device reuniting a violet light emission, three-dimensional imaging system, sound system and data transmission system.

US2014126787 to Lasarow Healthcare discloses a device and a computer-implemented method for generating a melanomy risk assessment. The asymmetry, border, and colour (ABC) of a mole is measured. The mole diameter information is used to determine whether an estimated diameter of the particular mole exceeds a predetermined threshold to provide diameter (D) analysis data. A melanoma risk assessments for the particular mole based on at least the ABC analysis data and the D analysis data is generated. However, this document does not disclose a temperature sensor, nor a calibration.

U.S. Ser. No. 10/674,953 to Welch Allyn discloses an imaging device for monitoring a skin feature includes a camera, at least one processor and memory. The imaging device displays a body area guide, receives a selection of a body area, initiates an image sequence of the body area, the image sequence including capturing, using the camera, a plurality of images, determines whether all body areas have been imaged, analyses the images, and provides the analysed images to a clinician. The imaging device can be part of a system including a server in communication with a monitor, where the clinician views the images on the monitor. Additionally, each skin feature can be ranked in order of risk and presented to the clinician in that order. A multispectral system may use multiple discrete sources, each of which illuminates the skin at a different wavelength, for example, 470 nm, 591 nm, 631 nm, and 850 nm for blue, yellow, red, and infrared, respectively. A broadband source may be used with filters that allow only the wavelengths of interest. The filter may be manually or automatically selected for each wavelength band of interest. A cooled detector allows the system to expand to mid- and far-infrared wavelengths. However, this imaging device does not comprise a temperature sensor nor a violet light emission source nor filters.

EP3000394 to Pixience discloses a colorimetric calibration device comprising a set of colorimetric zones of predetermined colours and at least one first location target located in the vicinity of said se of colorimetric zones. A patient films a dermatological abnormality present on his arm using his computer's video camera. The video stream from the camera is advantageously transmitted to a dermatologist who can observe the dermatological abnormality from a distance and make a diagnosis. However, the device does not disclose a temperature sensor.

WO2020240047 to Barco discloses a method and system for performing colour calibration of light sources and correction towards a colour target, for example a white point target. The calibration and correction considers the ambient temperature and that the projector output has an arbitrary dimming level. The correction can be made automatic and can be performed by an operator in the field by pressing a button. But this system does not disclose a device with a camera nor a violet light emission source.

US20140316235A1 to Digimark discloses a skin imaging application. A user uploads a new image of an unknown skin condition to the library. Image analysis techniques are employed to identify salient similarities between features of the uploaded image, and features of images in this reference library.

### SHORT DESCRIPTION OF THE INVENTION

The present inventors surprisingly have found that a multi-function device (100) allows for ergonomic remote monitoring of skin conditions. The multi-function device (100) captures three-dimensional image information, fluorescence information, temperature information and voice information. In a preferred embodiment, the multi-function device (100) calibrates varying light conditions, distances between the camera and the region of interest (200) of the skin, and angles through a calibration tube (10) mounted on the camera (20) of the multi-function device (100).

Accordingly, a first aspect of the invention is a multi-function device (100) for the ergonomic remote monitoring a medical or a cosmetic skin condition, comprising
A camera (20) comprising one or more objective lenses;
A detector configured to acquire a plurality of images of a region of interest (200) of the skin;
An adaptable light source
A temperature sensor for measuring the temperature of the skin;
A voice recording system (40);
A processor configured to produce from the image information obtained by the one or more imaging systems a multilayer three dimensional reconstruction of the region of interest (200) of the skin;
A graphical user interface (30) configured to display the image information and to receive instructions of the user via a touch screen;
A wireless communication system to transmit the image information obtained by the detector; and
An energy source;

In another embodiment, the multi-function device (100) is configured to
Register a calibration image (50) of the calibration region (55) in proximity of the region of interest (200) through the calibration tube (10) at a calibration distance;
Register a panoramic image (60) of the panoramic region (65) at a panoramic distance;
Register a near-distance image (70) of the region of interest (200) at a near distance
Fuse the calibration image (50), the panoramic image (60) and the near distance image (70) to obtain a calibrated superposed multi-layer reconstruction of the region of interest (200);
Transmitting the calibrated superposed multi-layer reconstruction of the region of interest (200) is transmitted through a secured connection (305, 315, 325, 335) in the cloud (300) to a user (310, 320, 330);
wherein the panoramic region (65) comprises the region of interest (200) and the calibration region (55);
and wherein the near-distance image (70) fully or partially excludes the calibration region (55).

In another embodiment, the calibration tube (10) of the multi-function device (100) is an opaque or partially or fully transparent hollow body.

In another embodiment, the calibration image (50) is an image of the tissue surrounding the region of interest (200).

In another embodiment, the panoramic image (60) comprises the skin tissue corresponding to the calibrating image (50) and the region of interest (200).

In another embodiment, the calibration tube (10) has a length of 1 cm or more, preferably of 2.5 cm or more, even more preferably of 5 cm or more.

In another embodiment, the calibration tube (10) has a length of 15 cm or less, preferably of 10 cm or less, even more preferably of 7.5 cm or less.

In another embodiment, the multi-function device (100) further the adaptable light source is an LED.

In another embodiment, the one or more light sources are infrared or violet light emission sources.

In another embodiment, the multi-function device (100) further comprises a voice recording system (40).

In another embodiment, the multi-function device (100) further comprises one or more filters comprising a plurality of apertures.

In another embodiment, the multi-function device (100) further comprises a sensor for measuring the temperature or the presence and quantity of bacteria or any other bodies in the region of interest (200).

Another aspect is a system for ergonomically and remotely monitoring a medical or a cosmetic skin condition, comprising
A multi-function device (100) according to any of the preceding claims;
A cloud-based data transmission system (300) configured to transmit the information obtained by the multi-function device (100) through a secured connection to a receiver (310, 320, 330);
One or more receivers (310, 320, 330) configured to receiving the information obtained by the multi-function device (100).

In another embodiment of the system of the invention, the multi-function device (100) is configured to
Registering a calibration image (50) of the calibration region (55) in proximity of the region of interest (200) through the calibration tube (10) at a calibration distance
Registering a panoramic image (60) of the panoramic region (65) at a panoramic distance
Registering a near-distance image (70) of the region of interest (200) at a near distance;
Fusing the calibration image (50), the panoramic image (60) and the near-distance image (70) to obtain a calibrated superposed multi-layer reconstruction of the region of interest (200);
Transmitting the calibrated superposed multi-layer reconstruction of the region of interest (200) is through a secured connection (305, 315, 325, 335) in the cloud (300) to a receiver (310, 320, 330)
wherein the panoramic region (65) comprises the region of interest (200) and the calibration region (55);
and wherein the near-distance image (70) fully or partially excludes the calibration region (55).

In another embodiment, the multi-function device (100) is configured to obtain a calibration image (50) with defined imaging conditions at a calibration distance. The calibration image (50) is an image of the calibration region (55). The calibration region (55) neighbors the region of interest (200). However, the calibration image (50) does not comprise the region of interest (200).

In another embodiment, the multi-function device (100) is further configured to obtain a panoramic image (60) of the panoramic region (65). The panoramic region (65) is obtained at a distance large enough to register both the calibration region (55) and the region of interest (200). A near-distance image (70) is obtained at a near distance. The near-distance image (70) does not comprise or only partially comprise the calibration region (55) corresponding to the calibration image (50). The near distance image (70) contains information corresponding to the near-distance region (75). The image data may be used to reconstruct a calibrated superposed multi-layer image of the region of interest (200).

### SHORT DESCRIPTION OF THE DRAWINGS

FIG. 1 shows a schematic illustration of the multi-function device (100) of the invention comprising a calibration tube (10) to a obtain a calibration image (50).

FIG. 2 shows a schematic illustration of the multi-function device (100) and a panoramic image (60) of the region of interest (200) that comprises the region of the skin corresponding to the calibration image (50).

FIG. 3 shows a schematic illustration of the multi-function device (100) and a near-distance image (70) of the region of interest (200) but not, or only partially the calibration region (55).

FIG. 4 shows a side view of the multi-function device (100) of the invention comprising a calibration tube (10) mounted on the camera (20) to a obtain a calibration image (50).

FIG. 5 shows a front view of the multi-function device (100) of the invention without the calibration tube (10) showing exemplary sensors (90).

FIG. 6 shows a rear view of the multi-function device (100) of the invention with a graphical user interface (30) and a voice recording system (40).

FIG. 7 shows a diagonal view of multi-function device (100) of the invention comprising a calibration tube (10).

FIG. 8 shows a schematic flow chart of the remote monitoring system (400) of the present invention comprising an multi-function device (100) of the invention comprising a calibration tube (10), a cloud based secured data transmission system (300, 305, 315, 325, 335) and one or more users (310, 320, 330).

### DETAILED DESCRIPTION OF THE DRAWINGS

Multi-Function Device (100)

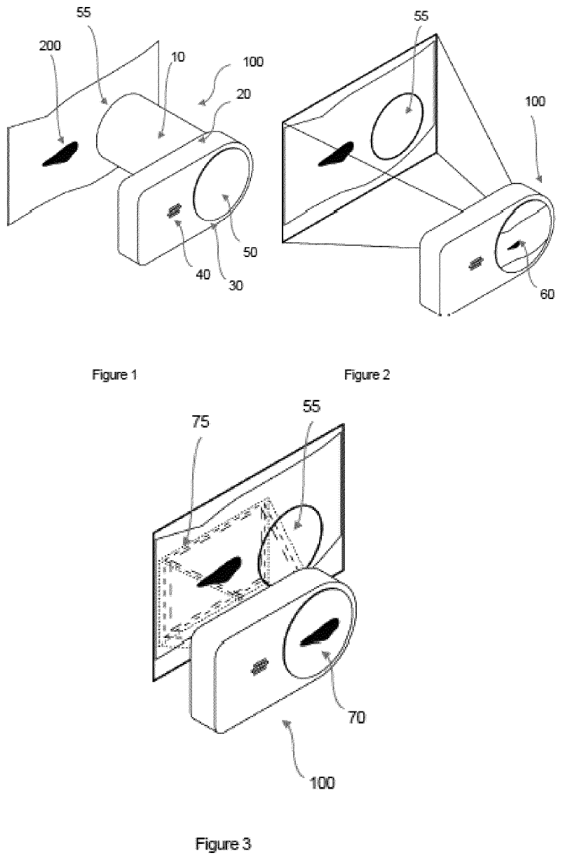

FIG. 1, FIG. 2 and FIG. 3 show an exemplary multi-function device (100) of the invention comprising:
A camera (20) comprising one or more objective lenses;
A detector configured to acquire a plurality of images of a region of interest (200) of the skin;
A processor configured to produce from the image information obtained by the one or more imaging systems a multilayer three dimensional reconstruction of the region of interest (200) of the skin;
A graphical user interface (30) to display the image information;
A wireless communication system to transmit the image information obtained by the detector; and
An energy source.

A calibration tube (10) is mounted on the camera (20) of the multi-function device (100). The multi-function device (100) is configured to obtain a calibration image (50) of the calibration region (55) in proximity of the region of interest (200) through the calibration tube (10) at a calibration distance as shown in FIG. 1. The calibration region (55) usually covers parts of the surrounding skin tissue of the region of interest (200). However, it does not comprise the region of interest (200).

The multi-function device (100) is further configured to obtain a panoramic image (60) of the panoramic region (65) at a panoramic distance as shown in FIG. 2.

The multi-function device (100) is further configured to obtain a near-distance image (70) of the region of interest (200) at a near distance as shown in FIG. 3. The near distance image (70) fully or partially excludes the calibration region (55).

The multi-function device (100) is further configured to fuse the calibration image (50) of FIG. 1, the panoramic image (60) of FIG. 2 and the near-distance image (70) of FIG. 3 to obtain a calibrated superposed multi-layer reconstruction of the region of interest (200).

FIG. 7 shows a diagonal view of multi-function device (100) of the invention comprising a calibration tube (10) mounted on the camera (20).

The multi-function device (100) is useful in remotely monitoring a region of interest (200) of the skin, preferably a lesion, a pathological tissue or wound, or the surrounding tissue.

Camera (20)

The camera (20) may be any suitable camera. In one embodiment, the camera is capable of registering a three-dimensional image of the region of interest (200).

Calibration Tube (10) for Controlled Environment

The calibration tube (10) enables a controlled environment for the registration of the calibration image (50). Controlled environment is understood as a region under the calibration tube (10) in which the brightness can be adjusted, hereinafter referred to as calibration region (55).

The calibration tube (10) may be opaque or fully or partially transparent. The image registered under calibration tube (10) serves as a reference for the calibration of the near-distance image (70). Calibration depends on the type of test pattern used or on the digital reconstruction. The calibration image (10) is obtained at a calibration distance, a calibration angle and a calibration lighting condition.

The calibration tube (10) is fixed to the camera (20) of the multi-function device (100) in a way to form a tunnel from the optics in the camera towards the calibration area (55) of the skin. The fixation of the calibration tube (10) to the camera of the multi-function device (100) may be permanent, rotatable or removable. In one embodiment, the calibration tube (10) is removable, cleanable and re-usable.

In one embodiment, the length of the calibration tube (10) corresponds to the calibration distance. In one embodiment, the calibration distance is 1 cm or more, preferably of 2.5 cm or more, even more preferably of 5 cm or more. In another embodiment, the calibration distance is 15 cm or less, preferably of 10 cm or less, even more preferably of 7.5 cm or less.

In one embodiment, the calibration tube (10) is extendable.

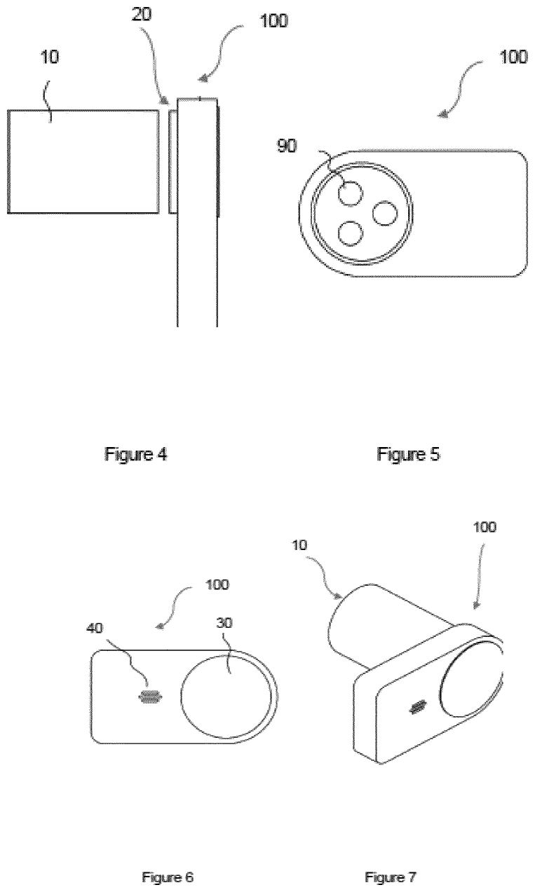

In another embodiment, the calibration tube (10) is removably or rotatably attached to the multi-function device (100). FIG. 4 shows a side view of the multi-function device (100) of the invention, wherein the calibration tube (10) is detached from the camera (20) for cleaning purposes.

The form of the calibration tube (10) may vary. In one embodiment, the calibration tube (10) is round or in the form of a hollow cylinder. However, the calibration tube (10) may be any other suitable hollow body. For example the calibration tube (10) may have a polygonal, quadrangular or rectangular cross section. The calibration tube (10) may have a symmetrical cross section or an asymmetrical cross section. The cross section may taper off or may widen.

Calibration

The multi-function device (100) is further configured to fuse the calibration image (50), the panoramic image (60) and the near-distance image (70) to obtain a calibrated superposed multi-layer reconstruction of the region of interest (200). This fusion step may be performed in the multi-function device (100) or remotely, for example in a cloud system, or for example in a protected medical cloud system.

In one embodiment, the calibration is performed through machine learning, wherein one or more of the calibration image (50), the panoramic image (60), and the near-distance image (70) are deformed.

In another embodiment, convolutional neural networks are used for the deformation of the calibration image (50), the panoramic image (60), and the near-distance image (70). The network is trained on the calibration image (50), the panoramic image (60), and the near-distance image (70) of a region of interest (200). Typically, for training purposes, the region of interest (200) is a wound or a lesion that is monitored over time.

Sensors (90)

The multi-function device (100) may have one or more sensors (90) for measuring other parameters of the region of interest (200) for example fluorescence, temperature or for measuring the presence, type or quantity of bacteria or other bodies. Exemplary sensors are shown in FIG. 5.

In one embodiment, the sensor usage is detected via magnetic connectors.

In another embodiment, the position of the multi-function device (100) is detected via an accelerometer.

Violet Light Emission and Filters

the multi-function device (100) further comprises a violet light emission source.

In another embodiment, the violet light is 100 nm to 600 nm, preferably 200 nm to 500 nm and even more preferably 350 nm to 450 nm. Violet light is used to detect bacteria and other features of interest, for example moist, blood, dirt or external bodies in the region of interest (200).

In Another Embodiment, a Filter is Mounted on the Camera (20). Graphical User Interfase (30)

The graphical user interphase (30) may be any system capable of displaying image information. The graphical user interface is configured to receive instructions from the user through a touch screen.

Voice Recording System (40)

The multi-function device (100) has a voice recording system (40). The voice recording system (40) enables the reconstruction of voice messages that may be transmitted through the wireless communication system. Consequently, medical and cosmetic professionals and practitioners may provide remote consultations on a medical or cosmetic skin condition based on the image and the voice information obtained and transmitted by the multi-function device (100).

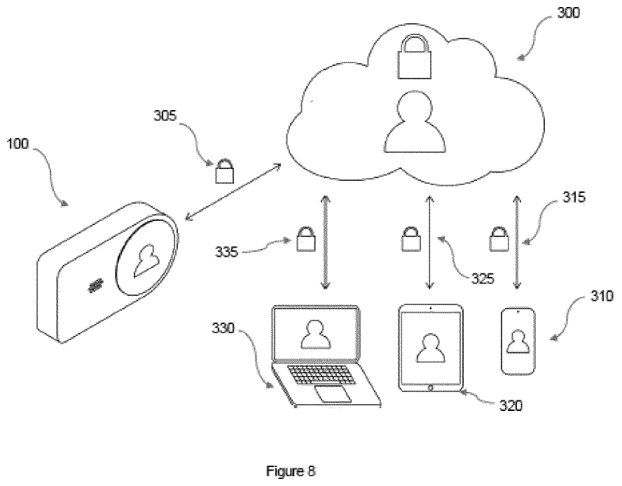

FIG. 6 shows a rear view of the multi-function device (100) of the invention with a graphical user interface (30) and a voice recording system (40).

Wireless Communication System

The wireless communication system may be any wireless communication system capable of transmitting the image information. The wireless communication system allows for example for medical and cosmetic professionals and practitioners to remotely obtain a calibrated image of a region of interest (200), in particular a medical or cosmetic skin condition.

Three-Dimensional Imaging and Reconstruction of Skin Lesion

The multi-function device (100) is particular useful in three-dimensional imaging and reconstruction of images of skin lesions or wounds.

Image Reconstruction Method

The image reconstruction method of the invention comprises the following steps:
Providing an multi-function device (100) of the invention;
Obtaining a calibration image (50) of the calibration region (55) in proximity of the region of interest (55) through the calibration tube (10) at a calibration distance
Obtaining a panoramic image (60) of the panoramic region (65) at a panoramic distance;
Obtaining a near-distance image (70) of the region of interest (200) at a near distance;
Fusing the calibration image (50), the panoramic image (60) and the near-distance image (70) of the region of interest (200) to obtain a calibrated superposed multi-layer reconstruction of the region of interest (200);
wherein the panoramic region (75) comprises the region of interest (200) and the calibration region (55); and
wherein the near-distance image (70) is configured to fully or partially exclude the calibration region (55).

In another embodiment, the image reconstruction method further comprises the step of obtaining the temperature of the region of interest (200) through a temperature sensor in the multi-function device (100).

In another embodiment, the image reconstruction method further comprises the step of obtaining a filtered image through a violet light emission source and filters mounted on the camera (20) of the multi-function device (100).

The image reconstruction method enables a medical and cosmetic professionals to obtain a calibrated superposed multilayer image, temperature of the region of interest (200) and voice information in respect of a medical or cosmetic skin condition remotely and in real time.

Remote Monitoring and Surveillance of a Medical or Cosmetic Skin Condition

FIG. 8 shows an exemplary remote monitoring system (400) of the present invention comprising an multi-function device (100) of the invention comprising a calibration tube (10), a cloud based secured data transmission system (300, 305, 315, 325, 335) and one or more users (310, 320, 330).

Accordingly, the multi-function device (100), image reconstruction method and image reconstruction system may be used to remotely monitor a region of interest (200), in particular a medical or cosmetic skin condition. Monitoring a medical or cosmetic skin condition means providing information at different points in time about the healing process, the evolution or development of a medical or cosmetic skin condition or the effectiveness of a treatment of a medical or cosmetic skin condition. Exemplary medical or cosmetic skin conditions include lesions, wounds or any other pathological conditions of the skin. It may also be used to support treatment decisions.

1. A multi-function device (100) for the ergonomic remote monitoring of a medical or a cosmetic skin condition, comprising
A camera (20) comprising one or more objective lenses;
A detector configured to acquire a plurality of images of a region of interest (200) of the skin;
An adaptable light source;
A temperature sensor for measuring the temperature of the skin;
A voice recording system (40);
A processor configured to produce from the image information obtained by the one or more imaging systems a multilayer three dimensional reconstruction of the region of interest (200) of the skin;
A graphical user interface (30) configured to display the image information and to receive instructions of the user via a touch screen;
A wireless communication system to transmit the image information obtained by the detector; and
An energy source.

2. The multi-function device (100) according to claim 1, wherein the multi-function device is configured to
Register a calibration image (50) of the calibration region (55) in proximity of the region of interest (200) through the calibration tube (10) at a calibration distance;
Register a panoramic image (60) of the panoramic region (65) at a panoramic distance;
Register a near-distance image (70) of the region of interest (200) at a near distance;
Fuse the calibration image (50), the panoramic image (60) and the near-distance image (70) to obtain a calibrated superposed multi-layer reconstruction of the region of interest (200);
Transmitting the calibrated superposed multi-layer reconstruction of the region of interest (200) is transmitted through a secured connection (305, 315, 325, 335) in the cloud (300) to a user (310, 320, 330);
wherein the panoramic region (65) comprises the region of interest (200) and the calibration region (55);
and wherein the near-distance image (70) fully or partially excludes the calibration region (55).

3. The multi-function device (100) according to claim 1, wherein the calibration tube (10) is an opaque or partially or fully transparent hollow body.

4. The multi-function device (100) according to claim 1, wherein the calibration image (50) is an image of the tissue surrounding the region of interest (200).

5. The multi-function device (100) according to claim 1, wherein the panoramic image (60) comprises the skin tissue corresponding to the calibrating image (50) and the region of interest (200).

6. The multi-function device (100) according to claim 1, wherein the calibration tube (10) has a length of 1 cm or more, preferably of 2.5 cm or more, even more preferably of 5 cm or more.

7. The multi-function device (100) according to claim 1, wherein the calibration tube (10) has a length of 15 cm or less, preferably of 10 cm or less, even more preferably of 7.5 cm or less.

8. The multi-function device (100) according to claim 1, wherein the multi-function device (100) further the adaptable light source is an LED.

9. The multi-function device (100) according to claim 1, wherein the one or more light sources are infrared or violet light emission sources.

10. The multi-function device (100) according to claim 1, wherein the multi-function device (100) further comprises a voice recording system (40).

11. The multi-function device (100) according to claim 1, wherein the multi-function device (100) further comprises one or more filters comprising a plurality of apertures.

12. The multi-function device (100) according to claim 1, wherein the multi-function device (100) further comprises a sensor for measuring the temperature or the presence and quantity of bacteria or any other bodies in the region of interest (200).

13. System for ergonomically and remotely monitoring a medical or a cosmetic skin condition, comprising
a. A multi-function device (100) according to claim 1;
b. A cloud-based data transmission system (300) configured to transmit the information obtained by the multi-function device (100) through a secured connection to a receiver (310, 320, 330);
c. One or more receivers (310, 320, 330) configured to receiving the information obtained by the multi-function device (1000);
wherein the multi-function device (100) is configured to
Registering a calibration image (50) of the calibration region (55) in proximity of the region of interest (200) through the calibration tube (10) at a calibration distance;
Registering a panoramic image (60) of the panoramic region (65) at a panoramic distance;
Registering a near-distance image (70) of the region of interest (200) at a near distance;
Fusing the calibration image (50), the panoramic image (60) and the near-distance image (70) to obtain a calibrated superposed multi-layer reconstruction of the region of interest (200);
Transmitting the calibrated superposed multi-layer reconstruction of the region of interest (200) is through a secured connection (305, 315, 325, 335) in the cloud (300) to a receiver (310, 320, 330);
wherein the panoramic region (65) comprises the region of interest (200) and the calibration region (55); and
wherein the near-distance image (70) fully or partially excludes the calibration region (55).

14. (canceled)
